SEQUENCE LISTING (6 sequences)

SEQ ID NO: 1 - DNA, 1200 bp, synthetic construct
  source 1..1200: mol_type = other DNA
gtgctcggccatcggctgagatcacgctgggaggatcagatgaccatcaaccctttcgaattccgcaccgttccctccatgcaggttgcctggggtggcgcccagcgcctcggagagattctttccggcaggtttcaggcgcgcaaggcgctgctgattaccgatgcaggtctcatcaaggccggcctcatcaagccgattgctgacgcacttgcggccagcggctttgacgtcacgatcttcgacaaggtcgttgctgatccgccagagcacatcgttgccgactgcgtcgagtccgcaaggcagatcggagtcgatatcgttattggtcttggcgggggctcatcgctcgacatcgccaagctcgtggctgtccttctggtttccgaccagacgcttgccgacatgtacgggatcggaaacgttaagggatcacgcctgccgctcgtgctggtgccgaccacggccggtaccggttcggaagtcacaaacatctccatcatcaccacgggagaaaccaccaagatgggggtcgtttctccgcagctttacgctgatttcgttcttctggatgctgaactgacggtgggactgccgcaggtccatacggctgccaccggtatcgatgccatggtgcatgccatcgaggcttacaccagcaaacacaagaagaacccgctatcagatgctctggcgcgcgaggcactgcggctgctcggtgcaaacctgatcgccgcctgcaggaacggggccgaccggaaagcccgtgaaggcatgcttttgggtgcgacattggcaggtcaggctttcgccaactcaccggtggccgccgtccacgcgctcgcttatccgctgggcggccattatcacgtgccacacgggctttccaatgccctgatgctggggccggttttacgctttaacgcgaaggctgcagcgtcgctttatgcggagcttgccgacgtgctgggtgttccgggtgaaggggatgcggcaacccgttcggatgcgttcgttcagcatatggaaacgctgatggacgaaagcggcgcgccgcgacgtctgcgcgatgtcggcgtgacggacaacacgctcgccatgcttgcgtccgacgcaatgaaacagagccgtctgttggtcaataatccggtcgaagtccgcgaagaggatgcgcttgcgctctaccgcgaggcgttctga

SEQ ID NO: 2 - protein, 399 aa, synthetic construct
MLGHRLRSRWEDQMTINPFEFRTVPSMQVAWGGAQRLGEILSGRFQARKALLITDAGLIKAGLIKPIADALAASGFDVTIFDKVVADPPEHIVADCVESARQIGVDIVIGLGGGSSLDIAKLVAVLLVSDQTLADMYGIGNVKGSRLPLVLVPTTAGTGSEVTNISIITTGETTKMGVVSPQLYADFVLLDAELTVGLPQVHTAATGIDAMVHAIEAYTSKHKKNPLSDALAREALRLLGANLIAACRNGADRKAREGMLLGATLAGQAFANSPVAAVHALAYPLGGHYHVPHGLSNALMLGPVLRFNAKAAASLYAELADVLGVPGEGDAATRSDAFVQHMETLMDESGAPRRLRDVGVTDNTLAMLASDAMKQSRLLVNNPVEVREEDALALYREAF

SEQ ID NO: 3 - DNA, 1413 bp, synthetic construct
  source 1..1413: mol_type = other DNA
atggtagtaggagatttccctattgaaacagatactcttgtaattggtgcgggacctggcggctatgtagctgccatccgcgctgcacagcttggacaaaaagtaacagtcgttgaaaaagcaactcttggaggcgtttgtctgaacgttggatgtatcccttcaaaagcgctgatcaatgcaggtcaccgttatgagaatgcaaaacattctgatgacatgggaatcactgctgagaatgtaacagttgatttcacaaaagttcaagaatggaaagcttctgttgtcaacaagcttactggcggtgtagcaggtcttcttaaaggcaacaaagtagatgttgtaaaaggtgaagcttactttgtagacagcaattcagttcgtgttatggatgagaactctgctcaaacatacacgtttaaaaacgcaatcattgctactggttctcgtcctatcgaattgccaaacttcaaatatagtgagcgtgtcctgaattcaactggcgctttggctcttaaagaaattcctaaaaagctcgttgttatcggcggcggatacatcggaactgaacttggaactgcgtatgctaacttcggtactgaacttgttattcttgaaggcggagatgaaattcttcctggcttcgaaaaacaaatgagttctctcgttacacgcagactgaagaaaaaaggcaacgttgaaatccatacaaacgcgatggctaaaggcgttgaagaaagaccagacggcgtaacagttactttcgaagtaaaaggcgaagaaaaaactgttgatgctgattacgtattgattacagtaggacgccgtccaaacactgatgagcttggtcttgagcaagtcggtatcgaaatgacggaccgcggtatcgtgaaaactgacaaacagtgccgcacaaacgtacctaacatttatgcaatcggtgatatcatcgaaggaccgccgcttgctcataaagcatcttacgaaggtaaaatcgctgcagaagctatcgctggagagcctgcagaaatcgattaccttggtattcctgcggttgttttctctgagcctgaacttgcatcagttggttacactgaagcacaggcgaaagaagaaggtcttgacattgttgctgctaaattcccatttgcagcaaacggccgcgcgctttctcttaacgaaacagacggcttcatgaagctgatcactcgtaaagaggacggtcttgtgatcggtgcgcaaatcgccggagcaagtgcttctgatatgatttctgaattaagcttagcgattgaaggcggcatgactgctgaagatatcgcaatgacaattcacgctcacccaacattgggcgaaatcacaatggaagctgctgaagtggcaatcggaagtccgattcacatcgtaaaataa

SEQ ID NO: 4 - protein, 470 aa, synthetic construct
MVVGDFPIETDTLVIGAGPGGYVAAIRAAQLGQKVTVVEKATLGGVCLNVGCIPSKALINAGHRYENAKHSDDMGITAENVTVDFTKVQEWKASVVNKLTGGVAGLLKGNKVDVVKGEAYFVDSNSVRVMDENSAQTYTFKNAIIATGSRPIELPNFKYSERVLNSTGALALKEIPKKLVVIGGGYIGTELGTAYANFGTELVILEGGDEILPGFEKQMSSLVTRRLKKKGNVEIHTNAMAKGVEERPDGVTVTFEVKGEEKTVDADYVLITVGRRPNTDELGLEQVGIEMTDRGIVKTDKQCRTNVPNIYAIGDIIEGPPLAHKASYEGKIAAEAIAGEPAEIDYLGIPAVVFSEPELASVGYTEAQAKEEGLDIVAAKFPFAANGRALSLNETDGFMKLITRKEDGLVIGAQIAGASASDMISELSLAIEGGMTAEDIAMTIHAHPTLGEITMEAAEVAIGSPIHIVK

SEQ ID NO: 5 - DNA, 627 bp, synthetic construct
  source 1..627: mol_type = other DNA
atgtctacagttttatttgtaaaatcaagcgaccgtacagctgaagaaggcgtttcaactaaactttacgaagctttcttagctgcttataaagaaaacaaccctaatgatgaagtggttgaattagatcttcataaggaaaaccttccttaccttggcagagatatgattaacggaacatttaaagcaggtcaaggaatggaaatgacagaagatgagaaaaaacaagcagcaattgctgacaaatatctgaaccagtttgtaaaagctgacaaagttgttttcgcattcccgctttggaacttcacagtgccagcagtgcttcatacttatgttgattatctgtctcgcgcaggcgttacattcaaatacacacaagaaggaccagtcggtttaatgggcggcaaaaaagttgcgcttcttaacgctcgcggcggtgtctactcagaaggaccaatggctgcacttgaaatgtcattaaacttcatgaaaacagttcttggtttctggggtgttcaagacttgcacacagttgtcatcgaaggacataacgcagcacctgatcaagcgcaagaaatcgttgaaaaaggtttacaagaagcaaaagatcttgctgcaaaattctaa

SEQ ID NO: 6 - protein, 208 aa, synthetic construct
MSTVLFVKSSDRTAEEGVSTKLYEAFLAAYKENNPNDEVVELDLHKENLPYLGRDMINGTFKAGQGMEMTEDEKKQAAIADKYLNQFVKADKVVFAFPLWNFTVPAVLHTYVDYLSRAGVTFKYTQEGPVGLMGGKKVALLNARGGVYSEGPMAALEMSLNFMKTVLGFWGVQDLHTVVIEGHNAAPDQAQEIVEKGLQEAKDLAAKF

(19) United States
(12) Patent Application Publication
(10) Pub. No.: US 2023/0408533 A1
(43) Pub. Date: Dec. 21, 2023

(54) RAPID ON-SITE DETECTION METHOD FOR GAMMA-HYDROXYBUTYRIC ACID

(71) Applicant: Northeastern University, Boston, MA (US)

(72) Inventors: Yunrong CHAI, Chestnut Hill, MA (US); Yuxuan Qin, Dorchester, MA (US); Nicole Taylor Cavanaugh, Chestnut Hill, MA (US)

(21) Appl. No.: 18/112,203

(22) Filed: Feb. 21, 2023

Related U.S. Application Data
(60) Provisional application No. 63/311,617, filed on Feb. 18, 2022.

(51) Int. Cl. G01N 33/94; G01N 21/78; C12N 9/04; C12N 9/02

(52) U.S. Cl. CPC G01N 33/9406; G01N 21/78; C12N 9/0006; C12Y 101/01061; C12N 9/0051; C12Y 108/01004; G01N 2021/7759

(57) ABSTRACT

Disclosed are compositions and methods for detecting the presence or level of gamma-hydroxybutyric acid (GHB), or for diagnosing GHB toxicity or overdose.

### RELATED APPLICATIONS

This application claims the benefit of priority to U.S. Provisional Patent Application Ser. No. 63/311,617, filed Feb. 18, 2022, which is hereby incorporated by reference in its entirety.

### REFERENCE TO A SEQUENCE LISTING XML

This application contains a Sequence Listing which has been submitted electronically in XML format. The Sequence Listing XML is incorporated herein by reference. Said XML file, created on Jun. 22, 2023, is named NEX-07401_SL.xml and is 10,552 in size.

### BACKGROUND

Gamma-hydroxybutyric acid (GHB), a naturally occurring neurotransmitter and a popular psychoactive drug. GHB is often used for recreational purposes as a club drug, and in some instances leads to drug-facilitated sexual assault. GHB can cause severe cognitive dysfunction and even death when overdosed. Since 1990, there has been about 16,000 death cases associated with GHB overdose or its analogs worldwide and substantially more cases of drug-facilitated sex assaults (DFSA). The risks associated with GHB is in part due to the lack of quick on-site diagnostic solutions. Intoxicated individuals are often left unaided due to the lack of knowledge about the cause of the toxicity. In the current market, the diagnosis of GHB toxicity or overdose almost exclusively relies on advanced, costly research facility and laboratory equipment, such as mass spectrometry, and skilled technicians to operate the equipment and analyze the results. A quick and easy method to detect the level of GHB in patients is needed.

### SUMMARY

In some aspect, provided herein is a kit for detecting the presence or the level or concentration of GHB in a biological sample, comprising: (1) a recombinant GHB dehydrogenase (GHBDH) enzyme; (2) a recombinant NADH-specific diaphorase; and (3) a dye.

In some embodiments, the kit further comprises a standard colorimetric paper strip which uses different degrees of the color to indicate different concentrations of GHB. In some embodiments, the biological sample is a human biological sample. In some embodiments, the biological sample is a blood, a plasma, a serum, or a urine sample. In some embodiments, the kit further comprises a reaction buffer, e.g., Tris buffer, phosphate buffer, or MOPS buffer. In some embodiments, the kit further comprises one or more factors. In some embodiments, the one or more factors are selected from metals, NAD, ATP, or tetrazolium salts. In some embodiments, the recombinant GHB dehydrogenase (GHBDH) enzyme and/or the recombinant NADH-specific diaphorase is provided in a lyophilized form. In some embodiments, the recombinant GHB dehydrogenase (GHBDH) enzyme and/or the recombinant NADH-specific diaphorase is provided in a buffer solution.

In some aspects, provided herein is a method of detecting the presence or the level or concentration of GHB in a biological sample, comprising: (a) contacting the biological sample with a kit described herein; (b) comparing the color of the reaction mix to the standard colorimetric paper strip to determine the concentration of GHB in the biological sample.

In some aspects, provided herein is a method of detecting the presence or the level or concentration of GHB in a biological sample, comprising: (a) contacting the biological sample with: (1) a recombinant GHBDH enzyme; (2) a recombinant NADH-specific diaphorase; and (3) a dye; (b) comparing the color of the reaction mix to a standard colorimetric paper strip to determine the concentration of GHB in the biological sample.

In some aspects, provided herein is a method of diagnosing GHB overdose or toxicity in a subject, comprising: (a) providing a biological sample obtained from the subject; (b) contacting the biological sample with a kit described herein; (c) comparing the color of the reaction mix to the standard colorimetric paper strip to determine the concentration of GHB in the biological sample; and (d) comparing the concentration determined in step (c) to a control level; wherein if the concentration is higher than the control level, then the subject is experiencing GHB overdose or toxicity; and if the concentration is equal to or lower than the control level, then the subject is not experiencing GHB overdose or toxicity.

In some aspects, provided herein is a method of diagnosing GHB overdose or toxicity in a subject, comprising: (a) providing a biological sample obtained from the subject; (b) contacting the biological sample with: (1) a recombinant GHBDH enzyme; (2) a recombinant NADH-specific diaphorase; and (3) a dye; (b) comparing the color of the reaction mix to a standard colorimetric paper strip to determine the concentration of GHB in the biological sample; and (c) comparing the concentration determined in step (c) to a control level; wherein if the concentration is higher than the control level, then the subject is experiencing GHB overdose or toxicity; and if the concentration is equal to or lower than the control level, then the subject is not experiencing GHB overdose or toxicity.

In some embodiments, the control level is a basal level of GHB detected by the same method in a subject of the same species that is not experiencing GHB overdose or toxicity. In some embodiments, the standard colorimetric paper strip uses different degrees of the color to indicate different concentrations of GHB. In some embodiments, the methods described herein further comprise obtaining the biological sample from a subject prior to step (a). In some embodiments, the subject is a human. In some embodiments, the methods described herein further comprise removing cells and/or large debris from the biological samples prior to step (a). In some embodiments, the biological sample is a blood, a plasma, a serum, or a urine sample.

In some embodiments, the recombinant GHBDH enzyme used in the kits or methods described herein is derived from bacterium Agrobacterium tumefaciens, e.g., Agrobacterium tumefaciens C58. In some embodiments, the recombinant GHBDH enzyme is AttL. In some embodiments, the recombinant GHBDH enzyme comprises an amino acid sequence having at least 85%, 90%, 95%, 99%, or 100% identity to SEQ ID NO: 2. In some embodiments, the recombinant GHBDH enzyme comprises an amino acid sequence having 100% identity to SEQ ID NO: 2.

In some embodiments, the recombinant NADH-specific diaphorase used in the kits or methods described herein is derived from bacterium Bacillus subtilis, e.g., Bacillus subtilis PY79. In some embodiments, the recombinant NADH-specific diaphorase is YvaB (also known as AzoR2), YocJ (also known as AzoR1), or PdhD. In some embodiments, the recombinant NADH-specific diaphorase comprises an amino acid sequence having at least 85%, 90%, 95%, 99%, or 100% identity to SEQ ID NO: 4 or 6. In some embodiments, the recombinant NADH-specific diaphorase comprises an amino acid sequence having 100% identity to SEQ ID NO: 4 or 6.

In some embodiments, the recombinant GHBDH enzyme and/or the recombinant NADH-specific diaphorase used in the kits or methods described herein is produced from a recombinant E. coli strain. In some embodiments, the recombinant E. coli strain is an Escherichia coli strain BL21/DE3. In some embodiments, the recombinant GHBDH enzyme and/or the recombinant NADH-specific diaphorase is purified.

In some embodiments, the dye used in the kits or methods described herein is a tetrazolium salt.

In some aspects, provided herein is recombinant E. coli strain comprising a nucleic acid comprising a nucleotide sequence encoding GHBDH, and/or a nucleotide sequence encoding NADH-specific diaphorase.

In some embodiments, the recombinant E. coli strain is an Escherichia coli strain BL21/DE3. In some embodiments, the nucleic acid is a plasmid. In some embodiments, the plasmid has a backbone of a shuttle vector pET28a(+).

In some embodiments, the GHBDH enzyme is derived from bacterium Agrobacterium tumefaciens, e.g., Agrobacterium tumefaciens C58. In some embodiments, the GHBDH enzyme is AttL. In some embodiments, the GHBDH enzyme comprises an amino acid sequence having at least 85%, 90%, 95%, 99%, or 100% identity to SEQ ID NO: 2. In some embodiments, the GHBDH enzyme comprises an amino acid sequence having 100% identity to SEQ ID NO: 2. In some embodiments, the nucleotide sequence encoding GHBDH enzyme comprises a nucleotide sequence having at least 85%, 90%, 95%, 99%, or 100% identity to SEQ ID NO: 1. In some embodiments, the nucleotide sequence encoding GHBDH enzyme comprises a nucleotide sequence having 100% identity to SEQ ID NO: 1.

In some embodiments, the NADH-specific diaphorase is derived from bacterium Bacillus subtilis, e.g., Bacillus subtilis PY79. In some embodiments, the NADH-specific diaphorase is YvaB (also known as AzoR2), YocJ (also known as AzoR1), or PdhD. In some embodiments, the NADH-specific diaphorase comprises an amino acid sequence having at least 85%, 90%, 95%, 99%, or 100% identity to SEQ ID NO: 4 or 6. In some embodiments, the NADH-specific diaphorase comprises an amino acid sequence having 100% identity to SEQ ID NO: 4 or 6. In some embodiments, the nucleotide sequence encoding NADH-specific diaphorase comprises a nucleotide sequence having at least 85%, 90%, 95%, 99%, or 100% identity to SEQ ID NO: 3 or 5. In some embodiments, the nucleotide sequence encoding NADH-specific diaphorase enzyme comprises a nucleotide sequence having 100% identity to SEQ ID NO: 3 or 5.

### BRIEF DESCRIPTION OF FIGURES

FIG. 1 shows a schematic diagram of a coupled enzymatic reaction converting GHB to colorimetric formazans.

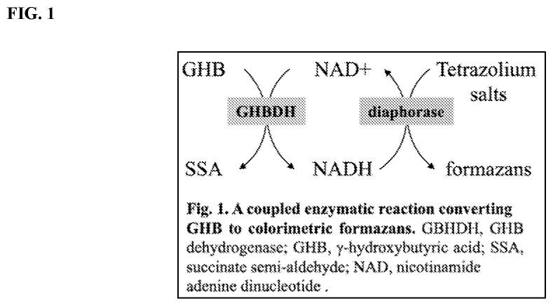

### DETAILED DESCRIPTION

### General

In some aspects, the present disclosure describes compositions and methods for a rapid on-site detection of gamma-hydroxybutyric acid (GHB), or a diagnosis of GHB toxicity or overdose. In some embodiments, the present disclosure provides a portable GHB detection solution for quick on-site GHB detection. The detection technology is based on, at least in part, coupled enzymatic reactions, converting GHB to a colorimetric substrate, so that the amount of GHB in the blood, urine, or other biological samples can be determined. These unique enzymes were originally discovered in bacteria and activities and efficacy confirmed in previous researches. Advantages of the described detection method also include rapid detection (20-30 min), cost-effectiveness, and easiness to use by individuals following simple instructions. The detection kits provided by the present disclosure have real-life applications. It will have a sizable market and visible social impact on addressing the health risk associated with GHB usage and drug-facilitated sexual assaults once the detection kits or methods described herein are widely adopted by clinics, urgent cares, law enforcement, and private sectors such as night clubs.

In some embodiments, this GHB detection method is based primarily on two recombinant enzymes of microbial sources. The first enzyme, a GHB dehydrogenase (GHBDH), originally from the bacterium Agrobacterium tumefaciens, converts GHB to succinate semialdehyde and concomitantly converts the reaction cofactor nicotinamide adenine dinucleotide (NAD) from the oxidized form to the reduced form (from NAD+ to NADH). It has previously been shown based on in vitro assays that the recombinant GHBDH enzyme demonstrated great efficacy and excellent kinetics in converting GHB to succinate semialdehyde (Chai et al. (2007) Journal of Bacteriology 189:3674-3679, the content of which is incorporated by reference herein in its entirety). The second enzyme in the coupled enzymatic reactions is a NADH specific diaphorase originally from the bacterium Bacillus subtilis, oxidizing NADH back to NAD while simultaneously converts tetrazolium salts to formazans. The latter are dyes that has a visible spectrum at 420-480 nm depending on specific dyes applied in the reaction. Through the coupled enzymatic reactions, the level of the produced colorimetric formazans correlates with NADH and thus GHB in the samples. When the color of the reaction solution is compared to a colorimetric paper strip that use different degrees of the color to indicate different concentrations of GHB, the original GHB levels in the blood or urine samples can be determined without sophisticated equipment.

The rapid on-site detection kits and methods provided herein have many different commercial applications, including but not limited to: (1) used by hospitals, diagnostic centers, urgent cares; (2) used by Law enforcement teams on the patrol; (3) used by Private sectors such as night clubs and bars; and (4) used as a commercial product (detection kit) for research purposes and other commercial applications. It can also be used by the research community in general.

Advantages of the compositions and methods provided herein include but are not limited to: (1) the concept of the detection method is novel and is based on coupled enzymatic reactions and uses a colorimetric assay as the readout; (2) portability is a key feature and the detection technology can be provided on-site; (3) the detection method is rapid and cost-effective compared to current technologies primarily based on advanced research equipment such as mass spectrometry; (4) the detection method can be operated by average individuals following simple instructions, no need for skilled technicians; (5) they provide rapid detection in the range of 20-30 min versus hours by current technology; and (6) they provide flexibility in modifying the detection method for different applications. Cost advantages include, e.g., the cost for purchasing such a detection kit is a small fraction of the expense to purchase, maintain, and perform assays on a mass spectrometry.

Advantages of the described detection kits and methods also include rapid detection, in the range of 20-30 min versus hours or even days of sending samples, and carrying out assays using mass spectrometry, and analyzing the results by trained scientists or skilled technicians. Another advantage of the described detection kits and methods include performing the detection on-site, no need to ship the samples to specialized labs or diagnostic centers.

### Definitions

The singular forms “a,” “an,” and “the” include the plurals unless the context clearly dictates otherwise.

The term “biological sample” as used herein refers to any sample of biological origin potentially containing one or more biomarker proteins (e.g., GHB). Examples of biological samples include tissue, organs, or bodily fluids such as whole blood, plasma, serum, urine, tissue, lavage or any other specimen used for detection of a disease or a condition (e.g., GHB overdose or toxicity).

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “comprise” and “comprising” are used in the inclusive, open sense, meaning that additional elements may be included.

The “basal” level of a marker (e.g., GHB) is the level of the marker (e.g., GHB) in a sample (e.g., a blood or urine sample) of a subject, e.g., a human, not afflicted with a disease or condition associated with aberrant marker levels (e.g., a subject not experiencing GHB overdose or toxicity).

The term “detecting” is used in the broadest sense to include both qualitative and quantitative measurements of a target molecule (e.g., GHB). In one embodiment, the detecting method as described herein is used to identify the mere presence of GHB in a biological sample. In another embodiment, the method is used to test whether GHB in in a sample is at a detectable level. In yet another embodiment, the method is used to test whether GHB in in a sample is at a toxic level. In still another embodiment, the method can be used to quantify the amount of GHB in a sample and further to compare the GHB levels from different samples, or from a control level (e.g., a basal level detected by the same method in a subject of the same species that is not experiencing GHB overdose or toxicity).

The terms “diagnosis” and “diagnostics” also encompass the terms “prognosis” and “prognostics”, respectively, as well as the applications of such procedures over two or more time points to monitor the diagnosis and/or prognosis over time, and statistical modeling based thereupon. Furthermore, the term diagnosis includes: a. prediction (determining if a patient will likely develop a disease or condition) b. prognosis (predicting whether a patient will likely have a better or worse outcome at a pre-selected time in the future) c. therapy selection d. therapeutic drug monitoring.

The term “gene” is used broadly to refer to any nucleic acid associated with a biological function. The term “gene” applies to a specific genomic sequence, as well as to a cDNA or an mRNA encoded by that genomic sequence.

The term “nucleic acid” or “nucleic acid molecule”, as used herein, is intended to include DNA molecules and RNA molecules. A nucleic acid molecule may be single-stranded or double-stranded, but preferably is double-stranded DNA.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

A “kit” is any manufacture (e.g., a package or container) comprising one or more reagents, e.g., recombinant enzymes and a dye described herein, for specifically detecting and/or affecting the expression of a marker (e.g., GHB) encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., GHB. Reagents in the kit may be provided in individual containers or as mixtures of two or more reagents in a single container. In addition, instructional materials which describe the use of the compositions within the kit may be included.

As used herein, a gene is “overexpressed” in a bacterium if it is expressed at a higher level in an engineered bacterium under at least some conditions than it is expressed by a wild-type bacterium of the same species under the same conditions. Similarly, a gene is “underexpressed” in a bacterium if it is expressed at a lower level in an engineered bacterium under at least some conditions than it is expressed by a wild-type bacterium of the same species under the same conditions.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

As used herein, the term “percent sequence identity” refers to the degree of identity between any given query sequence and a subject sequence. A percent identity for any query nucleic acid or amino acid sequence, relative to another subject nucleic acid or amino acid sequence can be determined as follows.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The terms “polynucleotide” and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

The term “providing” as used herein with regard to a biological sample refers to directly or indirectly obtaining the biological sample from a subject. For example, “providing” may refer to the act of directly obtaining the biological sample from a subject (e.g., by a blood draw, tissue biopsy, lavage and the like). Likewise, “providing” may refer to the act of indirectly obtaining the biological sample. For example, providing may refer to the act of a laboratory receiving the sample from the party that directly obtained the sample, or to the act of obtaining the sample from an archive.

The term “recombinant host cell” (or simply “host cell”), as used herein, is intended to refer to a cell that comprises a nucleic acid that is not naturally present in the cell, and maybe a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). For example, the subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject or patient may be healthy, may be GHB addictive or have GHB dependency, may be at risk of experiencing GHB overdose and/or toxicity, or may be suffering from GHB overdose and/or toxicity. In some embodiments, the subject has undergone a therapy that prevents, treats, and/or reduces severity of GHB overdose, toxicity, and/or addiction.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The term “vector,” as used herein, is intended to refer to a nucleic acid molecule (e.g., DNA or RNA) capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, vector also included are other forms of expression vectors, such as viral vectors (e.g., replication defective retroviruses, adenoviruses and adeno-associated viruses), which serve equivalent functions.

### Recombinant Enzymes

In some embodiments, the compositions and methods provided herein are based primarily on two recombinant enzymes of microbial sources. The first enzyme, a GHB dehydrogenase (GHBDH), e.g., AttL derived from the bacterium Agrobacterium tumefaciens, converts GHB to succinate semialdehyde and concomitantly converts the reaction cofactor nicotinamide adenine dinucleotide (NAD) from the oxidized form to the reduced form (from NAD+ to NADH). The second enzyme in the coupled enzymatic reactions is a NADH specific diaphorase, e.g., a YvaB, YocJ, or PdhD derived from the bacterium Bacillus subtilis, oxidizing NADH back to NAD while simultaneously converts tetrazolium salts to formazans. The chemistry of formazans and tetrazolium salts is further described in H. S̨enöz, Hacettepe J. Biol. & Chem., 2012, 40 (3), 293-301, the content of which is incorporated by reference herein in its entirety.

There are multiple dihydrolipoyl dehydrogenases (diaphorases) in Bacillus subtilis, all of which would work for the kits and methods described herein. In some embodiments, the NADH specific diaphorase is YvaB derived from the bacterium Bacillus subtilis. Additional description of YvaB can be found in Yoshiaki NishiYa & Yoshihiro Yamamoto (2007) Bioscience, Biotechnology, and Biochemistry, 71:2, 611-614, the content of which is incorporated by reference herein in its entirety. In some embodiments, the NADH specific diaphorase is YocJ derived from the bacterium Bacillus subtilis. Additional description of YocJ can be found in Leelakriangsak M. et al. (2008) Mol. Microbiol. 67(5):1108-1124, the content of which is incorporated by reference herein in its entirety. In some embodiments, the NADH specific diaphorase is PdhD derived from the bacterium Bacillus subtilis.

Representative amino acid and nucleotide sequences of GHB dehydrogenase (GHBDH) and NADH specific diaphorase are shown in Table 1 below:

TABLE 1
Sequence of attL gene (SEQ ID NO: 1)
GTGCTCGGCCATCGGCTGAGATCACGCTGGGAGGATCAGATGACCATCAACCCTTTCGAATT
CCGCACCGTTCCCTCCATGCAGGTTGCCTGGGGTGGCGCCCAGCGCCTCGGAGAGATTCTTT
CCGGCAGGTTTCAGGCGCGCAAGGCGCTGCTGATTACCGATGCAGGTCTCATCAAGGCCGGC
CTCATCAAGCCGATTGCTGACGCACTTGCGGCCAGCGGCTTTGACGTCACGATCTTCGACAA
GGTCGTTGCTGATCCGCCAGAGCACATCGTTGCCGACTGCGTCGAGTCCGCAAGGCAGATCG
GAGTCGATATCGTTATTGGTCTTGGCGGGGGCTCATCGCTCGACATCGCCAAGCTCGTGGCT
GTCCTTCTGGTTTCCGACCAGACGCTTGCCGACATGTACGGGATCGGAAACGTTAAGGGATC
ACGCCTGCCGCTCGTGCTGGTGCCGACCACGGCCGGTACCGGTTCGGAAGTCACAAACATCT
CCATCATCACCACGGGAGAAACCACCAAGATGGGGGTCGTTTCTCCGCAGCTTTACGCTGAT
TTCGTTCTTCTGGATGCTGAACTGACGGTGGGACTGCCGCAGGTCCATACGGCTGCCACCGG
TATCGATGCCATGGTGCATGCCATCGAGGCTTACACCAGCAAACACAAGAAGAACCCGCTAT
CAGATGCTCTGGCGCGCGAGGCACTGCGGCTGCTCGGTGCAAACCTGATCGCCGCCTGCAGG
AACGGGGCCGACCGGAAAGCCCGTGAAGGCATGCTTTTGGGTGCGACATTGGCAGGTCAGGC
TTTCGCCAACTCACCGGTGGCCGCCGTCCACGCGCTCGCTTATCCGCTGGGCGGCCATTATC
ACGTGCCACACGGGCTTTCCAATGCCCTGATGCTGGGGCCGGTTTTACGCTTTAACGCGAAG
GCTGCAGCGTCGCTTTATGCGGAGCTTGCCGACGTGCTGGGTGTTCCGGGTGAAGGGGATGC
GGCAACCCGTTCGGATGCGTTCGTTCAGCATATGGAAACGCTGATGGACGAAAGCGGCGCGC
CGCGACGTCTGCGCGATGTCGGCGTGACGGACAACACGCTCGCCATGCTTGCGTCCGACGCA
ATGAAACAGAGCCGTCTGTTGGTCAATAATCCGGTCGAAGTCCGCGAAGAGGATGCGCTTGC
GCTCTACCGCGAGGCGTTCTGA
AttL Amino Acid Sequence (SEQ ID NO: 2)
MLGHRLRSRWEDQMTINPFEFRTVPSMQVAWGGAQRLGEILSGRFQARKALLITDAGLIKAG
LIKPIADALAASGFDVTIFDKVVADPPEHIVADCVESARQIGVDIVIGLGGGSSLDIAKLVA
VLLVSDQTLADMYGIGNVKGSRLPLVLVPTTAGTGSEVTNISIITTGETTKMGVVSPQLYAD
FVLLDAELTVGLPQVHTAATGIDAMVHAIEAYTSKAKKNPLSDALAREALRLLGANLIAACR
NGADRKAREGMLLGATLAGOAFANSPVAAVHALAYPLGGHYHVPHGLSNALMLGPVLRFNAK
AAASLYAELADVLGVPGEGDAATRSDAFVQHMETLMDESGAPRRLRDVGVTDNTLAMLASDA
MKQSRLLVNNPVEVREEDALALYREAF
pdhD nucleotide sequence (SEQ ID NO: 3)
ATGGTAGTAG GAGATTTCCC TATTGAAACA GATACTCTTG TAATTGGTGC
GGGACCTGGC GGCTATGTAG CTGCCATCCG CGCTGCACAG CTTGGACAAA
AAGTAACAGT CGTTGAAAAA GCAACTCTTG GAGGCGTTTG TCTGAACGTT
GGATGTATCC CTTCAAAAGC GCTGATCAAT GCAGGTCACC GTTATGAGAA
TGCAAAACAT TCTGATGACA TGGGAATCAC TGCTGAGAAT GTAACAGTTG
ATTTCACAAA AGTTCAAGAA TGGAAAGCTT CTGTTGTCAA CAAGCTTACT
GGCGGTGTAG CAGGTCTTCT TAAAGGCAAC AAAGTAGATG TTGTAAAAGG
TGAAGCTTAC TTTGTAGACA GCAATTCAGT TCGTGTTATG GATGAGAACT
CTGCTCAAAC ATACACGTTT AAAAACGCAA TCATTGCTAC TGGTTCTCGT
CCTATCGAAT TGCCAAACTT CAAATATAGT GAGCGTGTCC TGAATTCAAC
TGGCGCTTTG GCTCTTAAAG AAATTCCTAA AAAGCTCGTT GTTATCGGCG
GCGGATACAT CGGAACTGAA CTTGGAACTG CGTATGCTAA CTTCGGTACT
GAACTTGTTA TTCTTGAAGG CGGAGATGAA ATTCTTCCTG GCTTCGAAAA
ACAAATGAGT TCTCTCGTTA CACGCAGACT GAAGAAAAAA GGCAACGTTG
AAATCCATAC AAACGCGATG GCTAAAGGCG TTGAAGAAAG ACCAGACGGC
GTAACAGTTA CTTTCGAAGT AAAAGGCGAA GAAAAAACTG TTGATGCTGA
TTACGTATTG ATTACAGTAG GACGCCGTCC AAACACTGAT GAGCTTGGTC
TTGAGCAAGT CGGTATCGAA ATGACGGACC GCGGTATCGT GAAAACTGAC
AAACAGTGCC GCACAAACGT ACCTAACATT TATGCAATCG GTGATATCAT
CGAAGGACCG CCGCTTGCTC ATAAAGCATC TTACGAAGGT AAAATCGCTG
CAGAAGCTAT CGCTGGAGAG CCTGCAGAAA TCGATTACCT TGGTATTCCT
GCGGTTGTTT TCTCTGAGCC TGAACTTGCA TCAGTTGGTT ACACTGAAGC
ACAGGCGAAA GAAGAAGGTC TTGACATTGT TGCTGCTAAA TTCCCATTTG
CAGCAAACGG CCGCGCGCTT TCTCTTAACG AAACAGACGG CTTCATGAAG
CTGATCACTC GTAAAGAGGA CGGTCTTGTG ATCGGTGCGC AAATCGCCGG
AGCAAGTGCT TCTGATATGA TTTCTGAATT AAGCTTAGCG ATTGAAGGCG
GCATGACTGC TGAAGATATC GCAATGACAA TTCACGCTCA CCCAACATTG
GGCGAAATCA CAATGGAAGC TGCTGAAGTG GCAATCGGAA GTCCGATTCA
CATCGTAAAA TAA
pdhD amino acid sequence (SEQ ID NO: 4)
MVVGDFPIET DTLVIGAGPG GYVAAIRAAQ LGQKVTVVEK ATLGGVCLNV
GCIPSKALIN AGHRYENAKH SDDMGITAEN VTVDFTKVQE WKASVVNKLT
GGVAGLLKGN KVDVVKGEAY FVDSNSVRVM DENSAQTYTF KNAIIATGSR
PIELPNFKYS ERVINSTGAL ALKEIPKKLV VIGGGYIGTE LGTAYANFGT
ELVILEGGDE ILPGFEKQMS SIVTRRLKKK GNVEIHTNAM AKGVEERPDG
VTVTFEVKGE EKTVDADYVL ITVGRRPNTD ELGLEQVGIE MTDRGIVKID
KQCRINVPNI YAIGDIIEGP PLAHKASYEG KIAAEAIAGE PAEIDYLGIP
AVVFSEPELA SVGYTEAQAK EEGLDIVAAK FPFAANGRAL SLNETDGFMK
LITRKEDGLV IGAQIAGASA SDMISELSLA IEGGMTAEDI AMTIHAHPTL
GEITMEAAEV AIGSPIHIVK
vocJ (also known as azok1) nucleotide sequence (SEQ ID NO: 5)
ATGTCTACAG TTTTATTTGT AAAATCAAGC GACCGTACAG CTGAAGAAGG
ATGTCTACAG TTTTATTTGT AAAATCAAGC GACCGTACAG CTGAAGAAGG
CGTTTCAACT AAACTTTACG AAGCTTTCTT AGCTGCTTAT AAAGAAAACA
ACCCTAATGA TGAAGTGGTT GAATTAGATC TTCATAAGGA AAACCTTCCT
TACCTTGGCA GAGATATGAT TAACGGAACA TTTAAAGCAG GTCAAGGAAT
GGAAATGACA GAAGATGAGA AAAAACAAGC AGCAATTGCT GACAAATATC
TGAACCAGTT TGTAAAAGCT GACAAAGTTG TTTTCGCATT CCCGCTTTGG
AACTTCACAG TGCCAGCAGT GCTTCATACT TATGTTGATT ATCTGTCTCG
CGCAGGCGTT ACATTCAAAT ACACACAAGA AGGACCAGTC GGTTTAATGG
GCGGCAAAAA AGTTGCGCTT CTTAACGCTC GCGGCGGTGT CTACTCAGAA
GGACCAATGG CTGCACTTGA AATGTCATTA AACTTCATGA AAACAGTTCT
TGGTTTCTGG GGTGTTCAAG ACTTGCACAC AGTTGTCATC GAAGGACATA
ACGCAGCACC TGATCAAGCG CAAGAAATCG TTGAAAAAGG TTTACAAGAA
GCAAAAGATC TTGCTGCAAA ATTCTAA
YocJ (also known as AzoR1) amino acid sequence (SEQ ID NO: 6)
MSTVLFVKSS DRTAEEGVST KLYEAFLAAY KENNPNDEVV ELDLHKENLP
YLGRDMINGT FKAGQGMEMT EDEKKQAAIA DKYLNQFVKA DKVVFAFPLW
NFTVPAVLHT YVDYLSRAGV TFKYTQEGPV GLMGGKKVAL LNARGGVYSE
GPMAALEMSL NFMKTVLGFW GVQDLHTVVI EGHNAAPDQA QEIVEKGLQE
AKDLAAKF

In some embodiments, the recombinant GHBDH enzyme used in the kits or methods described herein is derived from bacterium Agrobacterium tumefaciens, e.g., Agrobacterium tumefaciens C58. In some embodiments, the recombinant GHBDH enzyme is AttL. In some embodiments, the recombinant GHBDH enzyme comprises an amino acid sequence having at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 2. In some embodiments, the recombinant GHBDH enzyme comprises an amino acid sequence having 100% identity to SEQ ID NO: 2.

In some embodiments, the recombinant NADH-specific diaphorase used in the kits or methods described herein is derived from bacterium Bacillus subtilis, e.g., Bacillus subtilis PY79. In some embodiments, the recombinant NADH-specific diaphorase is YvaB, YocJ, or PdhD. In some embodiments, the recombinant NADH-specific diaphorase comprises an amino acid sequence having at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 4 or 6. In some embodiments, the recombinant NADH-specific diaphorase comprises an amino acid sequence having 100% identity to SEQ ID NO: 4 or 6.

In some embodiments, the recombinant enzyme described herein (e.g., GHBDH or diaphorase) comprises a tag. In some embodiments, the recombinant enzyme described herein (e.g., GHBDH or diaphorase) comprises an AVI tag, a Histidine tag, a Flag tag, and/or a Myc tag. In some embodiments, the recombinant enzyme described herein (e.g., GHBDH or diaphorase) is affinity purified.

In some embodiments, the recombinant GHBDH enzyme and/or the recombinant NADH-specific diaphorase used in the kits or methods described herein is produced from a recombinant E. coli strain. In some embodiments, the recombinant E. coli strain is an Escherichia coli strain BL21/DE3. In some embodiments, the recombinant GHBDH enzyme and/or the recombinant NADH-specific diaphorase is purified. Methods for purification of recombinant enzymes are well known in the art, including but not limited to, e.g., commercial kits for the affinity protein purification (e.g., Qiagen). The concentration and purify of the recombinant proteins can be determined by size-fractionation by SDS-PAGE, spectrometry, and/or Bradford assays.

### Vectors and Host Cells

Also provided are nucleotide sequences corresponding to (e.g., encoding) the GHBDH or NADH-specific diaphorase disclosed herein. These sequences include all degenerate sequences related to the disclosed antibodies, i.e., all nucleic acids having a sequence that encodes one particular peptide and variants and derivatives thereof. Thus, while each particular nucleic acid sequence may not be written out herein, it is understood that each and every sequence is in fact disclosed and described herein through the disclosed polypeptide sequences.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors can be either circular or linear. Such vectors may comprise regulatory elements, such as a promoter, enhancer, terminator and the like, to cause or direct expression of said polypeptide. The provided vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak; New Haven, CT) sequences typically are expressed as a fusion with the encoded polypeptide. Such tags can be inserted anywhere within the polypeptide including at either the carboxyl or amino terminus.

In some instances, the disclosure includes cells comprising the nucleic acids (e.g., vectors) and/or peptides disclosed herein. In some embodiments, the host cells are prokaryotic host cells, including E. coli cells, can be used as long as sequences requisite for maintenance in that host, such as appropriate replication origin(s), are present. For convenience, selectable markers are also provided. Host systems are known in the art and need not be described in detail herein. Prokaryotic host cells include bacterial cells, for example, E. coli. B. subtilis, and mycobacteria.

In general, cells that can be used herein are commercially available from, for example, the American Type Culture Collection (ATCC), P.O. Box 1549, Manassas, VA 20108. See also F. Ausubel et al., Current Protocols in Molecular Biology, John Wiley & Sons, New York, NY, (1998). Transformation and transfection methods useful in the generation of the cells disclosed herein are described, e.g., in F. Ausubel et al., Current Protocols in Molecular Biology, John Wiley & Sons, New York, NY, (1998).

In some aspects, provided herein is recombinant E. coli strain comprising a nucleic acid comprising a nucleotide sequence encoding GHBDH, and/or a nucleotide sequence encoding NADH-specific diaphorase.

In some embodiments, the recombinant E. coli strain is an Escherichia coli strain BL21/DE3. In some embodiments, the nucleic acid is a plasmid. In some embodiments, the plasmid has a backbone of a shuttle vector pET28a(+).

In some embodiments, the GHBDH enzyme is derived from bacterium Agrobacterium tumefaciens, e.g., Agrobacterium tumefaciens C58. In some embodiments, the GHBDH enzyme is AttL. In some embodiments, the GHBDH enzyme comprises an amino acid sequence having at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 2. In some embodiments, the GHBDH enzyme comprises an amino acid sequence having 100% identity to SEQ ID NO: 2.

In some embodiments, the nucleotide sequence encoding GHBDH enzyme comprises a nucleotide sequence having at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 1. In some embodiments, the nucleotide sequence encoding GHBDH enzyme comprises a nucleotide sequence having 100% identity to SEQ ID NO: 1.

In some embodiments, the NADH-specific diaphorase is derived from bacterium Bacillus subtilis, e.g., Bacillus subtilis PY79. In some embodiments, the NADH-specific diaphorase is YvaB, YocJ, or PdhD. In some embodiments, the NADH-specific diaphorase comprises an amino acid sequence having at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 4 or 6. In some embodiments, the NADH-specific diaphorase comprises an amino acid sequence having 100% identity to SEQ ID NO: 4 or 6.

In some embodiments, the nucleotide sequence encoding NADH-specific diaphorase comprises a nucleotide sequence having at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to SEQ ID NO: 3 or 5. In some embodiments, the nucleotide sequence encoding NADH-specific diaphorase enzyme comprises a nucleotide sequence having 100% identity to SEQ ID NO: 3 or 5.

### Kits

In some aspect, provided herein is a kit for detecting the presence or the level or concentration of GHB in a biological sample, comprising: (1) a recombinant GHB dehydrogenase (GHBDH) enzyme descried herein; (2) a recombinant NADH-specific diaphorase described herein; and (3) a dye.

In some embodiments, the dye used in the kits described herein is a tetrazolium salt. In some embodiments, the kit further comprises a standard colorimetric paper strip which uses different degrees of the color to indicate different concentrations of GHB.

In some embodiments, the kit further comprises a reaction buffer, e.g., Tris buffer, phosphate buffer, or MOPS buffer.

In some embodiments, the kit further comprises one or more factors, e.g., factors that facilitate the enzymatic reactions. In some embodiments, the one or more factors are selected from metals, NAD, ATP, or tetrazolium salts.

In some embodiments, the recombinant GHB dehydrogenase (GHBDH) enzyme and/or the recombinant NADH-specific diaphorase is provided in a lyophilized form.

In some embodiments, the recombinant GHB dehydrogenase (GHBDH) enzyme and/or the recombinant NADH-specific diaphorase is provided in a buffer solution.

In some embodiments, the kit may consist essentially of GHBDH, diaphorase, and a dye. In some embodiments, the kit contains these reagents in a solution, and the solution may comprise other species, such as peptides other than the GHBDH and diaphorase, lipids, polynucleotides, or other biological or non-biological species. In some embodiments, the solution may also contain other components to stabilize the solution. For instance, the solution may contain protein stabilizers and/or sodium azide. According to these embodiments, GHBDH, diaphorase, and the dye may all be as described herein.

In some embodiments, the kit further comprises purified GHB protein as a standard. In some embodiments, the kit further comprises a sample with GHB as a positive control, and/or a sample without GHB as a negative control.

In some embodiments, the kit may be promoted, distributed, or sold as a unit for performing the methods provided herein. Reagents in the kit may be provided in individual containers or as mixtures of two or more reagents in a single container. Reagents in the kit may be provided in an array.

A kit provided herein may also include instructional materials disclosing or describing the use of the kit. A kit may also include additional components to facilitate the particular application for which the kit is designed.

### Detection and Diagnosis Methods

In some aspects, provided herein is a method of detecting the presence or the level or concentration of GHB in a biological sample, comprising: (a) contacting the biological sample with a kit described herein; (b) comparing the color of the reaction mix to the standard colorimetric paper strip to determine the concentration of GHB in the biological sample.

In some aspects, provided herein is a method of detecting the presence or the level or concentration of GHB in a biological sample, comprising: (a) contacting the biological sample with: (1) a recombinant GHBDH enzyme; (2) a recombinant NADH-specific diaphorase; and (3) a dye; (b) comparing the color of the reaction mix to a standard colorimetric paper strip to determine the concentration of GHB in the biological sample.

In some aspects, provided herein is a method of diagnosing GHB overdose or toxicity in a subject, comprising: (a) providing a biological sample obtained from the subject; (b) contacting the biological sample with a kit described herein; (c) comparing the color of the reaction mix to the standard colorimetric paper strip to determine the concentration of GHB in the biological sample; and (d) comparing the concentration determined in step (c) to a control level; wherein if the concentration is higher than the control level, then the subject is experiencing GHB overdose or toxicity; and if the concentration is equal to or lower than the control level, then the subject is not experiencing GHB overdose or toxicity.

In some aspects, provided herein is a method of diagnosing GHB overdose or toxicity in a subject, comprising: (a) providing a biological sample obtained from the subject; (b) contacting the biological sample with: (1) a recombinant GHBDH enzyme; (2) a recombinant NADH-specific diaphorase; and (3) a dye; (b) comparing the color of the reaction mix to a standard colorimetric paper strip to determine the concentration of GHB in the biological sample; and (c) comparing the concentration determined in step (c) to a control level; wherein if the concentration is higher than the control level, then the subject is experiencing GHB overdose or toxicity; and if the concentration is equal to or lower than the control level, then the subject is not experiencing GHB overdose or toxicity.

In some embodiments, the dye used in the methods described herein is a tetrazolium salt. In some embodiments, the standard colorimetric paper strip uses different degrees of the color to indicate different concentrations of GHB.

In some embodiments, the control level is a basal level of GHB detected by the same method in a subject of the same species that is not experiencing GHB overdose or toxicity.

In some embodiments, the methods described herein further comprise obtaining the biological sample from a subject prior to step (a). In some embodiments, the subject is a human. In some embodiments, the methods described herein further comprise removing cells and/or large debris from the biological samples prior to step (a).

The term “biological sample” is intended to include tissues, cells, and biological fluids isolated from a subject, such as serum, as well as tissues, cells, and fluids present within a subject. That is, the detection method of the present invention can be used to detect GHB, in a biological sample in vitro as well as in vivo. In some embodiments, the biological sample is a blood, a plasma, a serum, or a urine sample.

### EXAMPLES

The development of GHB detection kits and methods can be divided into three stages. In the first stage, the two recombinant enzymes of microbial sources are produced.

To produce GHBDH enzyme, E. coli strain overexpressing GHBDH (AttL) has been built. Specifically, DNA sequences (or genes) encoding the GHBDH^{at} (attL) was amplified by polymerase chain reactions (PCR) from the source bacterium Agrobacterium tumefaciens C58. The DNA sequence encoding attL is shown in Table 2 below. The gene attL from Agrobacterium tumefaciens was then cloned into the vector pET28a under the T7 inducible phage promoter. This engineered plasmid is used for attK overexpression in the E. coli strain BL21. The promoter is inducible by the addition of IPTG.

To produce NADH-specific diaphorase, the DNA sequence encoding the NADH-specific diaphorase is amplified by PCR from the source bacterium Bacillus subtilis PY79. There are multiple dihydrolipoyl dehydrogenases (diaphorases) in Bacillus subtilis, all of which would work for the kits and methods described herein. In this particular example, PdhD derived from the bacterium Bacillus subtilis is used and the DNA sequence encoding PdhD is shown in Table 2 below. Diaphorase from Bacillus subtilis is then cloned into the vector pET28a under the T7 inducible phage promoter. This engineered plasmid is used for diaphorase overexpression in the E. coli strain BL21. The promoter is inducible by the addition of IPTG.

The amplified DNA sequences (GHBDH and diaphorase) are cloned into the shuttle vector [pET28a(+)] by established recombinant DNA techniques. The resulting recombinant plasmid successfully carrying the genes for GHBDH^{at}, and the diaphorase are verified by DNA sequencing. These recombinant plasmids are prepared in large quantities from the resulting Escherichia coli strain DH5α carrying each of the recombinant plasmids.

The recombinant plasmids generated above are subsequently introduced into another Escherichia coli strain BL21/DE3, a specially engineered strain to facilitate protein overexpression and affinity purification. The resulting recombinant strains of E. coli are used to produce recombinant enzymes of GHBDH^{at} and diaphorase. Overexpression and purification of these recombinant enzymes follow well-established protocols. Commercial kits are available to facilitate the affinity protein purification (e.g. Qiagen). The concentration and purify of the recombinant proteins are determined by size-fractionation by SDS-PAGE, spectrometry, and Bradford assays.

TABLE 2
Sequence of attL gene (SEQ ID NO: 1)
GTGCTCGGCCATCGGCTGAGATCACGCTGGGAGGATCAGATGACCATCAACCCTTTCGAATT
CCGCACCGTTCCCTCCATGCAGGTTGCCTGGGGTGGCGCCCAGCGCCTCGGAGAGATTCTTT
CCGGCAGGTTTCAGGCGCGCAAGGCGCTGCTGATTACCGATGCAGGTCTCATCAAGGCCGGC
CTCATCAAGCCGATTGCTGACGCACTTGCGGCCAGCGGCTTTGACGTCACGATCTTCGACAA
GGTCGTTGCTGATCCGCCAGAGCACATCGTTGCCGACTGCGTCGAGTCCGCAAGGCAGATCG
GAGTCGATATCGTTATTGGTCTTGGCGGGGGCTCATCGCTCGACATCGCCAAGCTCGTGGCT
GTCCTTCTGGTTTCCGACCAGACGCTTGCCGACATGTACGGGATCGGAAACGTTAAGGGATC
ACGCCTGCCGCTCGTGCTGGTGCCGACCACGGCCGGTACCGGTTCGGAAGTCACAAACATCT
CCATCATCACCACGGGAGAAACCACCAAGATGGGGGTCGTTTCTCCGCAGCTTTACGCTGAT
TTCGTTCTTCTGGATGCTGAACTGACGGTGGGACTGCCGCAGGTCCATACGGCTGCCACCGG
TATCGATGCCATGGTGCATGCCATCGAGGCTTACACCAGCAAACACAAGAAGAACCCGCTAT
CAGATGCTCTGGCGCGCGAGGCACTGCGGCTGCTCGGTGCAAACCTGATCGCCGCCTGCAGG
AACGGGGCCGACCGGAAAGCCCGTGAAGGCATGCTTTTGGGTGCGACATTGGCAGGTCAGGC
TTTCGCCAACTCACCGGTGGCCGCCGTCCACGCGCTCGCTTATCCGCTGGGCGGCCATTATC
ACGTGCCACACGGGCTTTCCAATGCCCTGATGCTGGGGCCGGTTTTACGCTTTAACGCGAAG
GCTGCAGCGTCGCTTTATGCGGAGCTTGCCGACGTGCTGGGTGTTCCGGGTGAAGGGGATGC
GGCAACCCGTTCGGATGCGTTCGTTCAGCATATGGAAACGCTGATGGACGAAAGCGGCGCGC
CGCGACGTCTGCGCGATGTCGGCGTGACGGACAACACGCTCGCCATGCTTGCGTCCGACGCA
ATGAAACAGAGCCGTCTGTTGGTCAATAATCCGGTCGAAGTCCGCGAAGAGGATGCGCTTGC
GCTCTACCGCGAGGCGTTCTGA
pdhD nucleotide sequence (SEQ ID NO: 3)
ATGGTAGTAG GAGATTTCCC TATTGAAACA GATACTCTTG TAATTGGTGC
ATGGTAGTAG GAGATTTCCC TATTGAAACA GATACTCTTG TAATTGGTGC
GGGACCTGGC GGCTATGTAG CTGCCATCCG CGCTGCACAG CTTGGACAAA
AAGTAACAGT CGTTGAAAAA GCAACTCTTG GAGGCGTTTG TCTGAACGTT
GGATGTATCC CTTCAAAAGC GCTGATCAAT GCAGGTCACC GTTATGAGAA
TGCAAAACAT TCTGATGACA TGGGAATCAC TGCTGAGAAT GTAACAGTTG
ATTTCACAAA AGTTCAAGAA TGGAAAGCTT CTGTTGTCAA CAAGCTTACT
GGCGGTGTAG CAGGTCTTCT TAAAGGCAAC AAAGTAGATG TTGTAAAAGG
TGAAGCTTAC TTTGTAGACA GCAATTCAGT TCGTGTTATG GATGAGAACT
CCTATCGAAT TGCCAAACTT CAAATATAGT GAGCGTGTCC TGAATTCAAC
CTGCTCAAAC ATACACGTTT AAAAACGCAA TCATTGCTAC TGGTTCTCGT
CCTATCGAAT TGCCAAACTT CAAATATAGT GAGCGTGTCC TGAATTCAAC
TGGCGCTTTG GCTCTTAAAG AAATTCCTAA AAAGCTCGTT GTTATCGGCG
GCGGATACAT CGGAACTGAA CTTGGAACTG CGTATGCTAA CTTCGGTACT
GAACTTGTTA TTCTTGAAGG CGGAGATGAA ATTCTTCCTG GCTTCGAAAA
ACAAATGAGT TCTCTCGTTA CACGCAGACT GAAGAAAAAA GGCAACGTTG
AAATCCATAC AAACGCGATG GCTAAAGGCG TTGAAGAAAG ACCAGACGGC
GTAACAGTTA CTTTCGAAGT AAAAGGCGAA GAAAAAACTG TTGATGCTGA
TTACGTATTG ATTACAGTAG GACGCCGTCC AAACACTGAT GAGCTTGGTC
TTGAGCAAGT CGGTATCGAA ATGACGGACC GCGGTATCGT GAAAACTGAC
AAACAGTGCC GCACAAACGT ACCTAACATT TATGCAATCG GTGATATCAT
CGAAGGACCG CCGCTTGCTC ATAAAGCATC TTACGAAGGT AAAATCGCTG
CAGAAGCTAT CGCTGGAGAG CCTGCAGAAA TCGATTACCT TGGTATTCCT
GCGGTTGTTT TCTCTGAGCC TGAACTTGCA TCAGTTGGTT ACACTGAAGC
ACAGGCGAAA GAAGAAGGTC TTGACATTGT TGCTGCTAAA TTCCCATTTG
CAGCAAACGG CCGCGCGCTT TCTCTTAACG AAACAGACGG CTTCATGAAG
CTGATCACTC GTAAAGAGGA CGGTCTTGTG ATCGGTGCGC AAATCGCCGG
AGCAAGTGCT TCTGATATGA TTTCTGAATT AAGCTTAGCG ATTGAAGGCG
GCATGACTGC TGAAGATATC GCAATGACAA TTCACGCTCA CCCAACATTG
GGCGAAATCA CAATGGAAGC TGCTGAAGTG GCAATCGGAA GTCCGATTCA
CATCGTAAAA TAA

In the second stage of the development of the GHB detection kits and methods, the formulation of the reaction mixtures are optimized. The following steps are performed:
1. Different reaction buffers commonly used in research (Tris buffer, phosphate buffer, MOPS buffer, etc.) are applied to determine the optimal reaction buffer for the detection assays based on the best kinetics of the reactions.
2. All factors (including metals, NAD, ATP, tetrazolium salts, etc.) important for the efficient enzymatic reactions and their optimal concentrations are determined based on optimal kinetics of the enzymatic reactions.
3. A lyophilized form of the two enzyme mixture are prepared following the established technique and its effectiveness is compared to enzyme mixtures prepared in the buffer solute. The purpose of this is to test if the lyophilized enzymes, which will significantly improve the storage and life-span of the enzymes, can be applied in the detection assay.

In the third stage of the method development, the goal is to incorporate important controls in order to eliminate the impact of the native GHB level in the blood or urine samples, as well as to establish a standard curve to correlate the colorimetric results of the assays with known concentrations of pure GHB applied in the assays. The following steps are performed:
1. Human body carries low, native levels of GHB from the body's own metabolism. It is important to provide this information using the detection method provided herein. Assays are carried out in blood and urine samples of average individuals obtained from hospital or diagnostic centers. The results determines the basal level of GHB detected by this method. A simple filtration step using a mini-syringe is applied to remove cells and large debris from the biological samples prior to the detection.
2. A standard curve that correlates the known GHB concentrations to specific results of the colorimetric assays is created and used to generate the colorimetric paper strip. This colorimetric paper strip shows different degrees of the color correlating different concentrations of GHB.

Upon the completion of the three stages discussed above, recombinant enzymes are successfully prepared, blood and urine samples are tested, a standard curve to correlate known GHB concentrations to the results of the colorimetric assays is provided, a standard colorimetric paper strip is generated, and important information about the basal body GHB levels using the detection method are provided. As a next step, a prototype product of a detection kit based on the above detection method is developed by working with industrial partners.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A kit for detecting the presence or the level or concentration of GHB in a biological sample, comprising:
(1) a recombinant GHB dehydrogenase (GHBDH) enzyme;
(2) a recombinant NADH-specific diaphorase; and
(3) a dye.

2. The kit of claim 1, wherein the recombinant GHBDH enzyme is derived from bacterium Agrobacterium tumefaciens; optionally wherein the recombinant GHBDH enzyme is derived from bacterium Agrobacterium tumefaciens C58.

3. The kit of claim 1, wherein the recombinant GHBDH enzyme is AttL.

4. The kit of claim 1, wherein the recombinant GHBDH enzyme comprises an amino acid sequence having at least 85%, 90%, 95%, 99%, or 100% identity to SEQ ID NO: 2.

5. (canceled)

6. The kit of claim 1, wherein the recombinant NADH-specific diaphorase is derived from bacterium Bacillus subtilis; optionally wherein the recombinant NADH-specific diaphorase is derived from bacterium Bacillus subtilis PY79.

7. The kit of claim 6, wherein the recombinant NADH-specific diaphorase is YvaB, YocJ, or PdhD.

8. The kit of claim 1, wherein the recombinant NADH-specific diaphorase comprises an amino acid sequence having at least 85%, 90%, 95%, 99%, or 100% identity to SEQ ID NO: 4 or 6.

9. (canceled)

10. The kit of claim 1, wherein the dye is a tetrazolium salt.

11. The kit of claim 1, further comprising a standard colorimetric paper strip which uses different degrees of the color to indicate different concentrations of GHB.

12. The kit of claim 1, wherein the biological sample is a human biological sample.

13. (canceled)

14. The kit of claim 1, further comprising a reaction buffer.

15. The kit of claim 14, wherein the reaction buffer is Tris buffer, phosphate buffer, or MOPS buffer.

16. The kit of claim 1, further comprising one or more factors.

17. The kit of claim 16, wherein the one or more factors are selected from metals, NAD, ATP, and tetrazolium salts.

18. The kit of claim 1, wherein the recombinant GHB dehydrogenase (GHBDH) enzyme and/or the recombinant NADH-specific diaphorase is provided in a lyophilized form.

19. The kit of claim 1, wherein the recombinant GHB dehydrogenase (GHBDH) enzyme and/or the recombinant NADH-specific diaphorase is provided in a buffer solution.

20. A method of detecting the presence or the level or concentration of GHB in a biological sample, comprising:
(a) contacting the biological sample with a kit of claim 1;
(b) comparing the color of the reaction mix to the standard colorimetric paper strip to determine the concentration of GHB in the biological sample.

21. A method of diagnosing GHB overdose or toxicity in a subject, comprising:
(a) providing a biological sample obtained from the subject;
(b) contacting the biological sample with a kit of claim 1;
(c) comparing the color of the reaction mix to the standard colorimetric paper strip to determine the concentration of GHB in the biological sample; and
(d) comparing the concentration determined in step (c) to a control level;
wherein if the concentration is higher than the control level, then the subject is experiencing GHB overdose or toxicity; and if the concentration is equal to or lower than the control level, then the subject is not experiencing GHB overdose or toxicity.

22. A method of detecting the presence or the level or concentration of GHB in a biological sample, comprising:
(a) contacting the biological sample with:
(1) a recombinant GHBDH enzyme;
(2) a recombinant NADH-specific diaphorase; and
(3) a dye;
(b) comparing the color of the reaction mix to a standard colorimetric paper strip to determine the concentration of GHB in the biological sample.

23.-41. (canceled)

42. A recombinant E. coli strain, comprising a nucleic acid comprising a nucleotide sequence encoding GHBDH, and/or a nucleotide sequence encoding NADH-specific diaphorase.

43.-57. (canceled)
